(19) United States
(12) Patent Application Publication
(10) Pub. No.: US 2005/0176045 A1
(43) Pub. Date: Aug. 11, 2005

(54) SNP discriminatory siRNA

(75) Inventors: Yuriy Fedorov, Superior, CO (US); Angela Reynolds, Conifer, CO (US); Anastasia Khvorova, Boulder, CO (US)

Correspondence Address: KALOW & SPRINGUT LLP, 488 MADISON AVENUE, 19TH FLOOR, NEW YORK, NY, 10022, US

(73) Assignee: Dharmacon, Inc., Lafayette, CO (US)

(21) Appl. No.: 11/043,637

(22) Filed: Jan. 25, 2005

Related U.S. Application Data
(60) Provisional application No. 60/542,669, filed on Feb. 6, 2004.
(60) Provisional application No. 60/543,663, filed on Feb. 10, 2004.

(51) Int. Cl. C12Q 1/68; C12N 15/85

(52) U.S. Cl. 435/6; 435/455

(57) ABSTRACT

A method of identifying SNP specific siRNA is provided. The method comprises comparing the silencing effect of: (i) at least two SNP containing siRNA in cells that contain a SNP target sequence; (ii) said at least two SNP containing siRNA in cells that contain a wild type target sequence; (iii) at least two non-SNP containing siRNA in cells that contain a SNP target sequence; and (iv) said at least two non-SNP containing siRNA in cells that contain a wild type target sequence. Through the method, SNP specific siRNA can be selected for a diverse set of genes, including the Kras gene.

### CROSS REFERENCE TO RELATED APPLICATIONS

This application claims the benefit of U.S. Provisional Application No. 60/542,669, filed Feb. 6, 2004, and U.S. Provisional Application No. 60/543,663, filed Feb. 10, 2004, each of which is incorporated herein by reference.

### FIELD OF INVENTION

The present invention relates to the detection and degradation of mRNA and to the blockage of translation or transcription of genes that contain Single Nucleotide Polymorphisms.

### BACKGROUND OF THE INVENTION

Relatively recently, researchers observed that double stranded RNA (“dsRNA”) could be used to inhibit protein expression. This ability to silence a gene has broad potential for treating human diseases, and many researchers and commercial entities are currently investing considerable resources in developing therapies based on this technology.

Double stranded RNA induced gene silencing can occur on at least three different levels: (i) transcription inactivation, which refers to RNA guided DNA or histone methylation; (ii) siRNA induced mRNA degradation; and (iii) mRNA induced transcriptional attenuation.

It is generally considered that the major mechanism of RNA induced silencing (“RNA interference,” or “RNAi”) in mammalian cells is mRNA degradation. Initial attempts to use RNAi in mammalian cells focused on the use of long strands of dsRNA. However, these attempts to induce RNAi met with limited success, due in part to the induction of the interferon response, which results in a general, as opposed to a target-specific, inhibition of protein synthesis. Thus, long dsRNA is not a viable option for RNAi in mammalian systems.

More recently it has been shown that when short (18-30 bp) RNA duplexes are introduced into mammalian cells in culture, sequence-specific degradation of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell.

From a mechanistic perspective, long double stranded RNA introduced into plants and invertebrate cells is broken down into siRNA by a Type III-like endonuclease activity in Dicer. Dicer processes the dsRNA into 19-23 base pair siRNAs with characteristic two base 3′ overhangs. The siRNAs are then incorporated into an RNA-induced silencing complex (RISC), where one or more helicases unwind the siRNA duplex, enabling the complementary antisense strand to guide target recognition. Upon binding to the appropriate target mRNA, one or more endonucleases within the RISC cleaves the target to induce silencing. Alternatively, these molecules may decrease gene expression by targeting particular genetic loci (e.g. genomic DNA) for silencing (e.g. by methylation).

The interference effect can be long lasting and may be detectable after many cell divisions. Moreover, RNAi can exhibit sequence specificity. Thus, the RNAi machinery can specifically knock down one type of transcript, while not affecting closely related mRNA. These properties make siRNA a potentially valuable tool for inhibiting gene expression, studying gene function, and performing drug target validation. Furthermore, siRNAs are potentially useful as therapeutic agents against: (1) diseases that are caused by over-expression or misexpression of genes; and (2) diseases brought about by expression of genes that contain mutations.

One type of known genetic anomaly is the Single Nucleotide Polymorphism (“SNP”). This refers to variance of one base from a wild type form of a gene. A SNP may cause the production of a mutated protein that is less functional than the wild type or non-functional compared to the wild type. Alternatively, a SNP could render a translated protein more active compared to the wild type, or to behave in a different manner. All of these consequences are potentially harmful to an organism. Thus, it would be desirable to be able to silence a SNP-containing gene through the relatively recently discovered technology of siRNA.

Unfortunately, many people possess not only a SNP containing gene but also a wild type gene. Thus, they are heterozygous. Accordingly, there is a need to develop a method to identify siRNA that can silence SNP-containing genes without impacting the translation of the wild type genes. The present invention provides a solution.

SUMMARY OF THE INVENTION

The present invention is directed to the silencing of SNP-containing genes.

According to one embodiment, the present invention provides a method of identifying SNP specific siRNA, said method comprising: (a) comparing the silencing effect of: (i) at least two SNP containing siRNA in cells that contain a SNP target sequence, (ii) said at least two SNP containing siRNA in cells that contain a wild type target sequence, (iii) at least two non-SNP containing siRNA in cells that contain a SNP target sequence, and (iv) said at least two non-SNP containing siRNA in cells that contain a wild type target sequence; and (b) identifying a SNP-specific siRNA that silences said SNP containing target sequence, but does not silence said wild type target sequence or identifying a non-SNP containing siRNA that silences said wild type target sequence, but does not silence said SNP containing target sequence.

According to a second embodiment, the present invention provides a method for silencing a SNP-containing target gene, said method comprising: exposing a SNP-containing double stranded polynucleotide (siRNA) comprising two separate strands or a unimolecular polynucleotide (shRNA) to a target nucleic acid, wherein said double stranded polynucleotide comprising two separate strands or unimolecular polynucleotide comprises an antisense strand and a sense strand.

According to a third embodiment, the present invention provides a method for silencing a SNP containing target sequence through use of a SNP-containing siRNA (including those of two separate strands or shRNA) that has been modified by a 2′-O-methyl modification on nucleotides 1 and 2 or 1, 2, and 3 at the 5′ end of the sense strand, and a 5′ phosphate group on the first nucleotide at the 5′ end of the antisense strand. Alternatively, said duplexes could contain 2′-O-methyl modification on nucleotides 1 and 2 or 1, 2, and 3 at the 5′ end of the sense strand, 2′-O-methyl modifications on nucleotides 1 and 2 or 1, 2, and 3 at the 5′ end of the antisense strand, and a 5′ phosphate group on the first nucleotide at the 5′ end of the antisense strand. As yet another alternative, the molecules could contain any of the previously described modifications plus 2′-O-methyl modifications on one or more of the Cs and Us of the sense strand and/or 2′-fluoro (Fl) modifications on one or more Cs and/or Us on the antisense strand.

According to a fourth embodiment, the present invention provides a polynucleotide that has a region that comprises the RNA sequence: SEQ. ID No. 1, GUUGGAGCUGUUGGCGUAGUU (sense strand), which down regulates Kras genes that contains a G→T alteration at nucleotide 35 (codon 12, also referred to as a G12V allele) of the open reading. The bold U refers to the SNP site. The polynucleotide may, for example, be part of a siRNA that contains two separate strands or a unimolecular polynucleotide such as a shRNA. Thus, it is preferably part of polynucleotide that either comprises two strands that form a duplex that is 18-30 base pairs in length, or part of unimolecular molecule that has a sense region (also referred to as a sense strand) and an antisense region (also referred to as a sense strand) that are capable of forming a duplex that is 18-30 base pairs in length. Preferably, the sense strand and antisense strand are substantially complementary, more preferably 100% complementary.

According to a fifth embodiment, the present invention provides a method for down regulating the expression of a mutant form of the human Kras gene. This method comprises administering a siRNA of the fourth embodiment to a cell or organism that is expressing or is capable of expressing the target gene

According to a sixth embodiment, the present invention provides a polynucleotide that comprises a region that has the sequence: SEQ. ID No. 2, 5′ GUUGGAGCUGGUGGCGUAGUU (sense strand), which exclusively down regulates wildtype Kras genes (G at position 35 of the open reading frame) but has no silencing effect on the G→T (mutant) version of the gene. The bold G represents the SNP site. The polynucleotide is preferably part of a polynucleotide that either comprises two separate strands that form a duplex that is 18-30 base pairs in length, or part of a unimolecular molecule that has a sense region and an antisense region that are capable of forming a duplex that is 18-30 base pairs in length. Preferably the sense strand and antisense strand in the case of double stranded polynucleotide and the sense region and antisense region in the case of a unimolecular polynucleotide are substantially complementary, more preferably 100% complementary.

According to a seventh embodiment, the present invention provides a method for down regulating the expression of the wildtype form of the human Kras gene. This method comprises administering a siRNA of the sixth embodiment to a target gene, or to a cell or organism that is expressing or is capable of expressing the target gene.

For a better understanding of the present invention together with other and further advantages and embodiments, reference is made to the following description taken in conjunction with the examples, the scope of which is set forth in the appended claims.

### BRIEF DESCRIPTION OF THE FIGURES

FIG. 1 is a histogram describing the silencing efficiency of a luciferase siRNA containing mismatches at each of the positions in the duplex. The Y-axis represents the level of expression presented as a percentage of a control (mock transfected cells). The X-axis represents the position of each base pair along the duplex and the nucleotide substitution used.

FIG. 2 is a histogram describing the silencing efficiency of a human cyclophilin B siRNA containing mismatches at each of the positions in the duplex. The Y-axis represents the level of expression presented as a percentage of a control (mock transfected cells). The X-axis represents the position of each base pair along the duplex and the nucleotide substitution used.

FIG. 3 is a histogram describing the silencing efficiency of wild type (black bars) and mutant (SNP) containing (white bars) siRNA (sequences in Table I and II) in cells that contain a wildtype form of Kras. The X-axis identifies the specific siRNA being used (oligos #1-20, or a SMART pool). The Y-axis shows the ratio of wildtype Kras expression to GAPDH (control gene) expression. The boxed region identifies an siRNA that is capable of distinguishing between mutant and wildtype forms of Kras.

FIG. 4 is a histogram describing the silencing efficiency of wild type (black bars) and mutant (SNP) containing (white bars) siRNA (sequences in Table I and II) in cells that contain the G12V form of Kras. The X-axis identifies the specific siRNA being used (oligos #1-20, or a SMART pool). The Y-axis shows the ratio of mutant Kras expression to GAPDH (control gene) expression. The boxed region identifies a siRNA that is capable of distinguishing between mutant and wildtype forms of Kras.

### DETAILED DESCRIPTION

### DEFINITIONS

Unless stated otherwise or suggested by context, the following terms and phrases have the meanings provided below:

Alkyl

The term “alkyl” refers to a hydrocarbyl moiety that can be saturated or unsaturated, and substituted or unsubstituted. It may comprise moieties that are linear, branched, cyclic and/or heterocyclic, and contain functional groups such as ethers, ketones, aldehydes, carboxylates, etc.

Exemplary alkyl groups include but are not limited to substituted and unsubstituted groups of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and alkyl groups of higher number of carbons, as well as 2-methylpropyl, 2-methyl-4-ethylbutyl, 2,4-diethylpropyl, 3-propylbutyl, 2,8-dibutyidecyl, 6,6-dimethyloctyl, 6-propyl-6-butyloctyl, 2-methylbutyl, 2-methylpentyl, 3-methylpentyl, and 2-ethylhexyl. The term alkyl also encompasses alkenyl groups, such as vinyl, allyl, aralkyl and alkynyl groups.

Substitutions within an alkyl group can include any atom or group that can be tolerated in the alkyl moiety, including but not limited to halogens, sulfurs, thiols, thioethers, thioesters, amines (primary, secondary, or tertiary), amides, ethers, esters, alcohols and oxygen. The alkyl groups can by way of example also comprise modifications such as azo groups, keto groups, aldehyde groups, carboxyl groups, nitro, nitroso or nitrile groups, heterocycles such as imidazole, hydrazino or hydroxylamino groups, isocyanate or cyanate groups, and sulfur containing groups such as sulfoxide, sulfone, sulfide, and disulfide.

Further, alkyl groups may also contain hetero substitutions, which are substitutions of carbon atoms, by for example, nitrogen, oxygen or sulfur. Heterocyclic substitutions refer to alkyl rings having one or more heteroatoms. Examples of heterocyclic moieties include but are not limited to morpholino, imidazole, and pyrrolidino.

2′ Carbon Modification

The phrase “2′ carbon modification” refers to a nucleotide unit having a sugar moiety, for example a deoxyribosyl moiety that is modified at the 2′ position. A “2′-O-alkyl modified nucleotide” is modified at this position such that an oxygen atom is attached both to the carbon atom located at the 2′ position of the sugar and to an alkyl group, e.g., 2′-O-methyl, 2′-O-ethyl, 2′-O-propyl, 2′-O-isopropyl, 2′-O-butyl, 2-O-isobutyl, 2′-O-ethyl-O-methyl (—OCH₂CH₂OCH₃), and 2′-O-ethyl-OH (—OCH₂CH₂OH). A “2′ carbon sense modification” refers to a modification at the 2′ carbon position of a nucleotide on the sense strand or within a sense region of polynucleotide. A “2′ carbon antisense modification” refers to a modification at the 2′ carbon position of a nucleotide on the antisense strand or within an antisense region of polynucleotide.

Complementary

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. “Substantial complementarity” in the context of this document refers to polynucleotide strands exhibiting 90% or greater complementarity, excluding regions of the polynucleotide strands, such as overhangs, that are selected so as to be noncomplementary. (“Substantial similarity” in the context of this document, refers to polynucleotide strands exhibiting 90% or greater similarity, excluding regions of the polynucleotide strands, such as overhangs, that are selected so as not to be similar.) Thus, for example, two polynucleotides of 29 nucleotide units each, wherein each comprises a di-dT at the 3′ terminus such that the duplex region spans 27 bases, and wherein 26 of the 27 bases of the duplex region on each strand are complementary, are substantially complementary since they are 96.3% complementary when excluding the di-dT overhangs.

Deoxynucleotide

The term “deoxynucleotide” refers to a nucleotide or polynucleotide lacking a hydroxyl group (OH group) at the 2′ and/or 3′ position of a sugar moiety. Instead, it has a hydrogen bonded to the 2′ and/or 3′ carbon. Within an RNA molecule that comprises one or more deoxynucleotides, “deoxynucleotide” refers to the lack of an OH group at the 2′ position of the sugar moiety, having instead a hydrogen bonded directly to the 2′ carbon.

Deoxyribonucleotide

The terms “deoxyribonucleotide” and “DNA” refer to a nucleotide or polynucleotide comprising at least one sugar moiety that has an H, rather than an OH, at its 2′ and/or 3′ position.

Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 90% or greater complementarity. For example, a mismatch in a duplex region consisting of 19 base pairs results in 94.7% complementarity, rendering the duplex region substantially complementary.

First 5′ Terminal Antisense Nucleotide

The phrase “first 5′ terminal antisense nucleotide” refers to the nucleotide of the antisense strand that is located at the 5′ most position of that strand with respect to the bases of the antisense strand that have corresponding complementary bases on the sense strand. Thus, in a double stranded polynucleotide that is made of two separate strands, it refers to the 5′ most base other than bases that are part of any 5′ overhang on the antisense strand. When the first 5′ terminal antisense nucleotide is part of a hairpin molecule, the term “terminal” refers to the 5′ most relative position within the antisense region and thus is the 5′ most nucleotide of the antisense region.

First 5′ Terminal Sense Nucleotide

The phrase “first 5′ terminal sense nucleotide” is defined in reference to the antisense nucleotide. In molecules that are comprised of two separate strands, it refers to the nucleotide of the sense strand that is located at the 5′ most position of that strand with respect to the bases of the sense strand that have corresponding complementary bases on the antisense strand. Thus, in a double stranded polynucleotide that is made of two separate strands, it is the 5′ most base other than bases that are part of any 5′ overhang on the sense strand. When the first 5′ terminal sense nucleotide is part of a unimolecular polynucleotide that is capable of forming a hairpin molecule, the term “terminal” refers to the relative position within the sense region as measured by the distance from the base complementary to the first 5′ terminal antisense nucleotide.

Gene Silencing

The phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference, a defined, though partially characterized pathway whereby siRNA act in concert with host proteins (e.g. the RNA induced silencing complex, RISC, or the RNA-induced Initiation of Transcriptional Gene Silencing, RITS) to degrade messenger RNA (mRNA) in a sequence-dependent fashion or affect gene expression by other pathways or mechanisms, including but not limited to epigenetic mechanisms such as DNA and/or histone methylation. The level of gene silencing can be measured by a variety of means, including, but not limited to, measurement of transcript levels by Northern Blot Analysis, B-DNA techniques, transcription-sensitive reporter constructs, expression profiling (e.g. DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g. fluorescent properties (e.g. GFP) or enzymatic activity (e.g. alkaline phosphatases), or several other procedures.

Halogen

The term “halogen” refers to an atom of fluorine, chlorine, bromine, iodine or astatine. The phrase “2′ halogen modified nucleotide” refers to a nucleotide unit having a sugar moiety that is modified with a halogen at the 2′ position, i.e. attached directly to the 2′ carbon position of the ribose or deoxyribose ring. The letters “F” or “Fl” refer to a fluorine.

Nucleotide

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O— and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Further, the term nucleotide also includes those species that have a detectable label, such as for example a radioactive or fluorescent moiety such as a fluorescent dye such as Cy3™ on the 5′ carbon of the ribose ring of the first terminal nucleotide of the sense strand, or mass label attached to the nucleotide.

Off-Target Silencing and Off-Target Interference

The phrases “off-target silencing” and “off-target interference” are defined as degradation of mRNA other than the intended target mRNA due to overlapping and/or partial homology by the siRNA sense or antisense strands with unintended secondary mRNA messages. Off-targeting can also be the result of siRNA interacting with unintended DNA or mRNA targets and affecting transcription or translation, respectively.

Polynucleotide

The term “polynucleotide” refers to polymers of nucleotides, and includes but is not limited to DNA, RNA, DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included.

Polyribonucleotide

The term “polyribonucleotide” refers to a polynucleotide comprising two or more modified or unmodified ribonucleotides and/or their analogs. The term “polyribonucleotide” is used interchangeably with the term “oligoribonucleotide.”

Ribonucleotide and Ribonucleic Acid

The term “ribonucleotide” and the phrase “ribonucleic acid” (RNA), refer to a modified or unmodified nucleotide or polynucleotide comprising at least one ribonucleotide unit. A ribonucleotide unit comprises an hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

Second 5′ Terminal Antisense Nucleotide

The phrase “second 5′ terminal antisense nucleotide” refers to the nucleotide that is immediately adjacent to the first 5′ terminal antisense nucleotide and attached to the 3′ position of the first 5′ terminal antisense nucleotide. Thus, it is the second most 5′ nucleotide of the antisense strand or region within the set of nucleotides for which there are corresponding sense nucleotides.

Second 5′ Terminal Sense Nucleotide

The phrase “second 5′ terminal sense nucleotide” refers to the nucleotide that is immediately adjacent to the first 5′ terminal sense nucleotide and attached to the 3′ position of the first 5′ terminal sense nucleotide. Thus, it is the second most 5′ nucleotide of the sense strand or region within the set of nucleotides for which there are corresponding antisense nucleotides.

siRNA

The term “siRNA” refers to small inhibitory RNA duplexes that induce the RNA interference (RNAi) pathway. These molecules can vary in length (generally between 18-30 base pairs) and contain varying degrees of complementarity to their target mRNA in the antisense strand. Some, but not all, siRNA have unpaired overhanging bases on the 5′ or 3′ end of the sense strand and/or the antisense strand. Unless otherwise specified, the term “siRNA” includes duplexes of two separate strands, as well as single strands that can form hairpin structures comprising a duplex region. In the context of the present invention, when referring to a siRNA, the phrases “antisense strand” and “sense strand” are used to refer to the portions that are to a certain degree complementary and homologous, respectively with the target sequence, and to encompass the regions of both siRNA that contain two separate strands and siRNA that are formed from unimolecular polynucleotides that are capable of forming hairpins.

siRNA may be divided into five (5) groups (non-functional, semi-functional, functional, highly functional, and hyper-functional) based on the level or degree of silencing that they induce in cultured cell lines. As used herein, these definitions are based on a set of conditions where the siRNA is transfected into said cell line at a concentration of 100 nM and the level of silencing is tested at a time of roughly 24 hours after transfection, and not exceeding 72 hours after transfection. In this context, “non-functional siRNA” are defined as those siRNA that induce less than 50% (<50%) target silencing. “Semi-functional siRNA” induce 50-79% target silencing. “Functional siRNA” are molecules that induce 80-95% gene silencing. “Highly-functional siRNA” are molecules that induce greater than 95% gene silencing. “Hyperfunctional siRNA” are a special class of molecules. For purposes of this disclosure, hyperfunctional siRNA are defined as those molecules that: (1) induce greater than 95% silencing of a specific target when they are transfected at subnanomolar concentrations (i.e., less than one nanomolar); and/or (2) induce functional (or better) levels of silencing for greater than 96 hours. These relative functionalities (though not intended to be absolutes) may be used to compare siRNAs to a particular target for applications such as functional genomics, target identification and therapeutics.

SmartPool

The term “SmartPool” refers to a group of two or more siRNA directed against a single target that have been identified as having a high degree of functionality using a rational design algorithm.

SNP Containing siRNA

The phrase “SNP containing siRNA” refers to an siRNA in which the antisense strand of the duplex is a sequence that is complementary to a target sequence that contains a SNP of interest. By contrast, a non-SNP containing siRNA would be one in which the antisense strand of the duplex is complementary to the wild type target sequence. Thus, at the SNP site, a non-SNP containing siRNA would contain the base complementary to the base located on the wild type target gene.

Target

The term “target” is used in a variety of different forms throughout this document and is defined by the context in which it is used. “Target mRNA” refers to a messenger RNA to which a given siRNA can be directed against. “Target sequence” and “target site” refer to a sequence to which the sense strand of a siRNA shows varying degrees of homology and the antisense strand exhibits varying degrees of complementarity. The term “siRNA target” can refer to the gene, mRNA, or protein against which an siRNA is directed. Similarly “target silencing” can refer to the state of a gene, or the corresponding mRNA or protein.

Transfection

The term “transfection” refers to a process by which agents are introduced into a cell. The list of agents that can be transfected is large and includes, but is not limited to, siRNA, sense and/or anti-sense sequences, DNA encoding one or more genes and organized into an expression plasmid, proteins, protein fragments, and more. There are multiple methods for transfecting agents into a cell including, but not limited to, electroporation, calcium phosphate-based transfections, DEAE-dextran-based transfections, lipid-based transfections, molecular conjugate-based transfections (e.g. polylysine-DNA conjugates), microinjection and others.

### Preferred Embodiments

The present invention is directed to gene silencing of genes that contain Single Nucleotide Polymorphisms. Through the use of the present invention, one is able to select siRNA that may be used to reduce the expression of a SNP containing gene while minimizing the effect on the expression of the wild type gene.

The present invention will now be described in connection with preferred embodiments. These embodiments are presented in order to aid in an understanding of the present invention and are not intended, and should not be construed, to limit the invention in any way. All alternatives, modifications and equivalents that may become apparent to those of ordinary skill upon reading this disclosure are included within the spirit and scope of the present invention.

Furthermore, this disclosure is not a primer on RNA interference or Single Nucleotide Polymorphisms. Basic concepts known to persons skilled in the art have not been set forth in detail.

According to a first embodiment, the present invention provides a method of identifying SNP specific siRNA. According to this method, one compares the silencing effect of: (i) at least two SNP containing siRNA in cells that contain a SNP target sequence; (ii) said at least two SNP containing siRNA in cells that contain a wild type target sequence; (iii) at least two non-SNP containing siRNA in cells that contain a SNP target sequence, and (iv) said at least two non-SNP containing siRNA in cells that contain a wild type target sequence. Based on the results of these empirical studies, one may identify a SNP specific siRNA that silences said SNP containing target sequence, but does not silence said wild type target sequence.

The amount of silencing is relative. Preferably the selected siRNA will be functional, more preferably highly-functional and most preferably hyperfunctional. However, one wants to select an siRNA that has both a satisfactory functionality and silencing differential over its effect on the corresponding wild type gene. For example, there may be more than one siRNA that silences the SNP containing target sequence, but that does not silence the wild type gene. In these cases, preferably, one will select the SNP containing siRNA that has the largest silencing differential over its effect on a corresponding wild type sequence, provided that the SNP containing siRNA has an acceptable level of silencing of the SNP containing target sequences, otherwise one may consider a SNP containing siRNA with not the highest, but nonetheless acceptable differential over the wild type (i.e., it still silences an acceptable amount of the SNP containing gene, but not an unacceptable amount of the wild type gene).

When selecting the potential SNP specific siRNA, one may try all SNP containing siRNA within a chosen size range, or a subset of those SNP containing siRNA that are selected (i) randomly; (ii) systematically by walking up or down the gene; or (iii) by using rationale design, as described for example in commonly owned patent application U.S. patent application Ser. No. 10/714,333, filed Nov. 14, 2003, and international patent application no. PCT/US2003/036787, filed Nov. 14, 2003, published on Jun. 3, 2004 as WO 2004/045543 A2, the entire disclosures of which are incorporated by reference herein. However, preferably one will test all SNP containing siRNA of a particular size for a particular target (e.g., all 19-mers, all 20-mers, all 21-mers, all 22-mers, or all 23-mers).

Preferably the siRNA that is selected will have no appreciable effect on the wild type target. More preferably it will have no effect on the wild type target. The phrase “no appreciable effect” refers to an effect that would not preclude the cell from normal functioning even if, for example, there were a small reduction in production of the protein at issue. Additionally, it should be noted that although typically a SNP is relatively less frequent in a population than a wild type variant, the technology of the present invention is equally applicable if one desires to silence the wild type and not the SNP.

Methods for testing potential siRNA in a cell and for measuring the silencing of a siRNA in a cell are well known to persons of ordinary skill in the art. They may be tested in a cell that expresses or is capable of expressing either the wild type gene or SNP containing gene exclusively, or in cells that are heterozygous and thus express or are capable of expressing both the wild type gene and SNP containing gene

The siRNA identified by the present invention may be used advantageously with diverse cell types, including but not limited to primary cells, germ cell lines and somatic cells. The cells may be stem cells or differentiated cells. For example, the cell types may be embryonic cells, oocytes, sperm cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes and cells of the endocrine or exocrine glands. Furthermore, the present invention is applicable for use for employing RNA interference against a broad range of genes, including but not limited to the 45,000 genes of human genome, such as those implicated in diseases such as diabetes, Alzheimer's and cancer, as well as all genes in the genomes of organisms including but not limited to humans, mice, rats, and others.

Furthermore, the polynucleotides of the present invention may be administered to a cell by any method that is now known or that comes to be known and that from reading this disclosure, one skilled in the art would conclude would be useful with the present invention. For example, the polynucleotides may be passively delivered to cells.

Passive uptake of modified polynucleotides can be modulated, for example, by the presence of a conjugate such as a polyethylene glycol moiety or a cholesterol moiety at the 5′ terminal of the sense strand and/or, in appropriate circumstances, a pharmaceutically acceptable carrier.

Other methods for delivery include, but are not limited to, transfection techniques employing DEAE-Dextran, calcium phosphate, cationic lipids/liposomes, microinjection, electroporation, immunoporation, and coupling of the polynucleotides to specific conjugates or ligands such as antibodies, antigens, or receptors.

Further, the method of assessing the level of gene silencing includes all methods that are now known or that come to be known, and that from reading this disclosure, one of ordinary skill would conclude would be useful in connection with the present invention. For example, the silencing ability of any given siRNA can be studied by one of any number of art tested procedures including but not limited to Northern analysis, Western Analysis, RT PCR, expression profiling, and others.

In some systems the expression of either or both the wild type and SNP containing target sequence in the absence of any siRNA may be too low to measure. In these cases, the effects of any given SNP-containing siRNA, including those with two separate strands or shRNA can be assessed by measuring the effects of said agents on one or more targets expressed from a reporter expression construct or a target gene expressed from an expression vector.

The present invention may be used in RNA interference applications that induce transient or permanent states of disease or disorder in an organism by, for example, attenuating the activity of a target nucleic acid of interest believed to be a cause or factor in the disease or disorder of interest. Target nucleic acids of interest can comprise genomic or chromosomal nucleic acids or extrachromosomal nucleic acids, such as viral nucleic acids.

Further, the present invention may be used in RNA interference applications that determine the function of a target nucleic acid or target nucleic acid sequence of interest. For example, RNA interference can be used to examine the effects of polymorphisms, such as biallelic polymorphisms, by attenuating the activity of a target nucleic acid of interest having one or the other allele, and observing the effect on the organism or system studied. Therapeutically, one allele or the other, or both, may be selectively silenced using RNA interference where selective allele silencing is desirable.

Still further, the present invention may be used in RNA interference applications, such as diagnostics, prophylactics, and therapeutics. For these applications, an organism suspected of having a disease or disorder that is amenable to modulation by manipulation of a particular target nucleic acid of interest is treated by administering siRNA. Results of the siRNA treatment may be ameliorative, palliative, prophylactic, and/or diagnostic of a particular disease or disorder. Preferably, the siRNA is administered in a pharmaceutically acceptable manner with a pharmaceutically acceptable carrier or diluent.

Therapeutic applications of the present invention can be performed with a variety of therapeutic compositions and methods of administration. Pharmaceutically acceptable carriers and diluents are known to persons skilled in the art. Methods of administration to cells and organisms are also known to persons skilled in the art. Dosing regimens, for example, are known to depend on the severity and degree of responsiveness of the disease or disorder to be treated, with a course of treatment spanning from days to months, or until the desired effect on the disorder or disease state is achieved. Chronic administration of siRNAs may be required for lasting desired effects with some diseases or disorders. Suitable dosing regimens can be determined by, for example, administering varying amounts of one or more siRNAs in a pharmaceutically acceptable carrier or diluent, by a pharmaceutically acceptable delivery route, and determining the amount of drug accumulated in the body of the recipient organism at various times following administration. Similarly, the desired effect (for example, degree of suppression of expression of a gene product or gene activity) can be measured at various times following administration of the siRNA, and this data can be correlated with other pharmacokinetic data, such as body or organ accumulation. Those of ordinary skill can determine optimum dosages, dosing regimens, and the like. Those of ordinary skill may employ EC₅₀ data from in vivo and in vitro animal models as guides for human studies.

Further, the polynucleotides can be administered in a cream or ointment topically, an oral preparation such as a capsule or tablet or suspension or solution, and the like. The route of administration may be intravenous, intramuscular, dermal, subdermal, cutaneous, subcutaneous, intranasal, oral, rectal, by eye drops, by tissue implantation of a device that releases the siRNA at an advantageous location, such as near an organ or tissue or cell type harboring a target nucleic acid of interest.

The above-described embodiment also enables one to identify positions where base pair mismatches have little or no effect on siRNA activity or the ability to discriminate between wild type and SNP-containing (or SNP-containing and WT) targets. Thus, for instance, as presented in Example 1 below, base pair mismatches at positions 1, 2, 3, 5, 13, and 14 had little or no effect on siRNA activity in the target studied. Knowledge of the positions of these “insensitive” sites can be important, particularly when one is dealing with targets that exhibit high degrees of variability (e.g., viral targets). Under some embodiments, it will be preferable to have a mismatch near the 5′ end of the sense strand.

According to a second embodiment, the present invention is directed to a method for silencing a SNP-containing target gene, said method comprising: exposing a SNP-containing siRNA to a target nucleic acid, wherein said SNP-containing siRNA comprises an antisense strand and a sense strand.

Pertaining to this embodiment, all of the conditions listed in the first embodiment, including those pertaining to cell types, methods of delivery, methods of detection, and applications are included within the spirit and scope of this embodiment.

Preferably the SNP containing double stranded polynucleotide (siRNA) of both the first and second embodiments comprises from 18-30 base pairs, more preferably from 19-25 base pairs, and most preferably from 19-23 base pairs, exclusive of overhangs. When a range such as 18-30 base pairs is provided, the range includes but is not limited to polynucleotides that contain 18 base pairs and polynucleotides that contain 30 base pairs. Preferably, the sense strand and antisense strand are substantially complementary over the range of base pairs, and more preferably 100% complementary over this range. Preferably the polynucleotide is RNA.

The double stranded polynucleotide may, when containing two separate strands, also contain overhangs at either the 5′ or 3′ end of either the sense strand or the antisense strand. However, preferably if there are any overhangs, they are only on the 3′ end of the sense strand and/or the antisense strand. Additionally, preferably any overhangs are six or fewer bases in length, more preferably two or fewer bases in length. Most preferably, there are either no overhangs, or overhangs of two bases on one or both of the sense strand and antisense strand.

Under conditions where the SNP containing molecule is unimolecular (i.e. an shRNA/ hairpin RNA) the hairpin is preferably organized in a fashion such that the antisense strand or region is upstream of a loop, and the sense strand or region is downstream of the loop. Thus, the antisense region is located on the 5′ side of the loop region, with the 3′ most part of the antisense region being the portion of the antisense region that is closest to the loop region. Similarly, the sense region is located on the 3′ side of the loop region, with the 5′ most part of the sense region being the portion of the sense region that is closest to the loop region. Preferably, the sense region and the antisense region are substantially complementary, more preferably 100% complementary.

When designing a unimolecular polynucleotide, specifically a left-handed unimolecular structure (e.g., 5′-AS-Loop-S) according to the present invention, preferably, the first 5′ terminal sense nucleotide is defined as the nucleotide that is the 18^{th}, 19^{th} or 20^{th} base of the sense region counting from the base that is complementary to the first 5′ terminal antisense nucleotide (i.e. counting from the 3′ end of the sense region). The first 5′ terminal sense nucleotide is defined in this manner because when unimolecular polynucleotides that are capable of forming hairpins enter a cell, typically, Dicer will process hairpin polynucleotides that contain lengthier duplex regions, into molecules that are comprised of two separate strands (siRNA) of approximately 18-20 base pairs, and it is desirable for these molecules to have the sense strand modifications associated with the end of this processed molecule. Most preferably, the first 5′ terminal sense nucleotide is defined as the nucleotide that is the 19^{th} base of the sense region from the 3′ end of the sense region. Further, preferably, the polynucleotide is capable of forming a left-handed hairpin.

The SNP-containing hairpin may be designed according to the parameters as described in commonly owned Provisional Patent Application Ser. No. 60/530133, filed Dec. 16, 2003. For example, the hairpin may comprise a loop structure, which preferably comprises from four to ten bases, an antisense region and a sense region, wherein the sense region and antisense regions are independently 19-23 base pairs in length and substantially complementary to each other. Preferable sequences of the loop structure include, for example, 5′-UUCG (SEQ. ID NO. 3), 5′-UUUGUGUAG (SEQ. ID NO. 4), and 5′-CUUCCUGUCA (SEQ. ID NO. 5). The hairpin RNA can be capable of forming a left hairpin or a right hairpin. Preferably, the hairpin is a left hairpin.

The shRNA can further comprise a stem region, wherein the stem region comprises one or more nucleotides or modified nucleotides immediately adjacent to the 5′ end and the 3′ end of the loop structure, and wherein the one or more nucleotides or modified nucleotides of the stem region are (or are not) target-specific. Preferably the entire length of the hairpin molecule is fewer than 100 bases, more preferably fewer than 85 bases. Additionally there may be overhangs at for example the 3′ end of the sense region.

According to a third embodiment, the present invention provides a method for silencing a SNP containing target sequence through use of a SNP-containing siRNA, (including siRNA with two separate strands and shRNA) that has been modified by a 2′-O-alkyl modification on nucleotides 1 and 2 or 1, 2, and 3 at the 5′ end of the sense strand, and a 5′ phosphate group on the first nucleotide at the 5′ end of the antisense strand. Alternatively, said duplexes could contain 2′-O-alkyl modifications on nucleotides 1 and 2 or 1, 2, and 3 at the 5′ end of the sense strand, 2′-O-alkyl modifications on nucleotides 1 and 2 or 1, 2, and 3 at the 5′ end of the antisense strand, and a 5′ phosphate group on the first nucleotide at the 5′ end of the antisense strand. As yet another alternative, the molecules could contain any of the previously described modifications plus additional 2′-O-alkyl modifications on one or more of the Cs and Us of the sense strand and/or 2′-fluoro (Fl) modifications on one or more Cs and/or Us on the antisense strand.

It is known that addition of chemical modifications to key positions along an RNA-RNA, RNA-DNA, or DNA-DNA duplex can significantly alter the chemical and functional properties of these molecules. The applicants appreciate that modifications can be added to the sense strand of a siRNA to prevent that strand from entering RISC and inducing sense strand specific off-target effects. Elimination of the sense strand from interactions with RISC can also alter the equilibrium of antisense strand-RISC interaction, thus improving the level of silencing by said antisense strand. Furthermore, the applicants recognize that modifications can be added to both the sense strand and the antisense strand to eliminate the off-target effects generated by both strands. Still further, modifications can be added to both strands that (in addition to eliminating off-target effects) can increase the stability of the duplex. These modifications may be used to the extent that they do not detract from the present invention. Examples of modifications of siRNA molecules are described in more detail in, e.g., commonly owned U.S. patent application Ser. No. 10/613,077, filed Jul. 1, 2003, published as U.S. 2004/0266707 Al on Dec. 30, 2004, the entire disclosure of which is incorporated by reference herein.

Preferably, the modification is attached to the 2′ position of the nucleotide's ribose ring (i.e. the 2′ carbon). According to the present embodiment, preferably the modification is a 2′-O-alkyl group. However, it may be any other modification that when used in the context of the present invention minimizes off-target effects by this strand. For example, the 2′ modified nucleotide may be selected from the group consisting of a 2′ halogen modified nucleotide, a 2′ amine modified nucleotide and a 2′ alkyl modified nucleotide if such modifications are included under conditions that do not detract from the efficiency of the molecule or improve the efficiency by e.g., minimizing off-target effects and/or increasing stability. Where the modification is a halogen, the halogen is preferably fluorine. Where the 2′ modified nucleotide is a 2′ amine modified nucleotide, the amine is preferably —NH₂. Where the 2′ modified nucleotide is a 2′-alkyl modification, preferably the modification is a 2′ methyl modification, wherein the carbon of the methyl moiety is attached directly to the 2′ carbon of the sugar moiety.

As noted above, preferably the modification is a 2′-O-alkyl group. More preferably the modification is selected from the group consisting of 2′-O-methyl, 2′-O-ethyl, 2′-O-propyl, 2′-O-isopropyl, 2′-O-butyl, 2′-O-isobutyl, 2′-O-ethyl-O-methyl (—OCH₂CH₂OCH₃), and 2′-O-ethyl-OH (—OCH₂CH₂OH). Most preferably, the 2′-O-alkyl modification is a 2′-O-methyl moiety. Further, there is no requirement that the modification be the same on each of the first 5′ terminal sense nucleotide and the second 5′ terminal sense nucleotide, and similarly on the first 5′ terminal antisense nucleotide and the second 5′ terminal antisense nucleotide when present. However, as a matter of practicality with respect to synthesizing the molecules of the present invention, it may be desirable to use the same modification throughout the siRNA.

Under certain embodiments there may also be a third 5′ terminal sense nucleotide and/or a third 5′ terminal antisense nucleotide that has a 2′ carbon modification, preferably a 2′-O-alkyl modification, and more preferably a 2′-O-methyl modification.

The 2′-O-alkyl modified SNP containing polynucleotide can comprise two separate strands or be unimolecular, and all of the conditions previously described as pertaining to cell type, methods of delivery, methods of detection, and applications are included within the spirit and scope of this embodiment.

Additionally, other modifications may be incorporated according to techniques that are now know or that come to be known and that from reading this disclosure, a person of ordinary skill would conclude would be beneficial for use in connection with the present invention.

For example, the applicants appreciate that new modifications and combinations may be discovered in the future that would assist in improving siRNA stability, efficiency, specificity and/or potency. Further, the modifications of certain embodiments of the present invention could be combined with modifications that are desired for other purposes. For example, in some instances, one modification could affect one particular step of off-target silencing (e.g. sense strand association with RISC) while a second modification could affect a completely different step e.g. altering the ability of sense/antisense strands to associate with targets that have less than 100% homology. Alternatively, two separate modifications could affect the same step. In some cases, two or more modifications could act additively or synergistically, limiting off-target effects by minimizing undesirable interactions or processes at one or more steps. In still other instances, one modification could eliminate off-target effects, but have detrimental consequences on more desirable properties, e.g., the potency or stability of the siRNA. In cases such as these, additional modifications could be added that restore functionality of the molecule.

Additionally stabilization modifications that are addressed to the phosphate backbone may be included in the polynucleotides for some applications of the present invention. For example, at least one phosphorothioate and/or methylphosphonate may be substituted for the phosphate group at some or all 3′ positions of any or all pyrimidines in the sense and/or antisense strands of the oligonucleotide backbone, as well as in any overhangs, loop structures or stem structures that may be present. Phosphorothioate (and methylphosphonate) analogues arise from modification of the phosphate groups in the oligonucleotide backbone. In the phosphorothioate, the phosphate O⁻ is replaced by a sulfur atom. In methylphosphonates, the oxygen is replaced with a methyl group. In one embodiment the phosphorothioate modification or methylphosphonate is located at the 3′ positions of all antisense strand nucleotides that also contain 2′ fluoro (or other halogen) modified nucleotides. Additionally, phosphorothioate 3′ modifications may be used instead of and independent of 2′ fluoro modifications to increase stability of an siRNA molecule. These modifications may be used in combination with the other modifications disclosed herein, or independent of those modifications in siRNA applications.

Nucleases typically use both the oxygen groups on the phosphate moiety and the 2′OH position of the ribose ring to mediate attack on RNA. Substitution of a sulfur group for one of the oxygens eliminates the ability of the phosphate to participate in this reaction, thus limiting the sensitivity of this site to nuclease digestion. However, it should be noted that phosphorothioates are typically toxic, thus, they would be beneficial primarily when any toxic effects are negated, which it is postulated might be accomplished by limiting the use of this modification to e.g., every other nucleotide, every third nucleotide, or every fourth nucleotide.

Furthermore, for some applications it may be desirable to incorporate a label into the nucleotides of the present invention, e.g., a fluorescent label, a radioactive label or a mass label.

Still further it may be desirable to use the sequences identified in connection with the present invention in combination with control, tracking or exaequo agents that contain the same sequences but also contain modifications. Certain modifications of siRNA are particularly useful in transforming an siRNA that is at least functional siRNA into an siRNA that is essentially non-functional, because for example, it is does not enter the RISC pathway. For example these combination may include: 2′ carbon modifications, preferably 2′-alkyl modifications of the first and second, (or first, second and third) sense and antisense nucleotides, 2′ carbon modifications, preferably 2′-alkyl modifications of at least one other sense nucleotide and at least one other antisense nucleotide, wherein the 5′ terminal first antisense nucleotide is not phosphorylated.

When using exaequo agents or controls, it may be desirable to modify the 5′ carbon position of the 5′ end of the sense and/or the antisense strand with a blocking group. The blocking group may for example be an alkyl group or any other group that prevents phosphorylation of the 5′ carbon position of the nucleotide. Phosphorylation may occur in a cell due to the activity of kinases that are present in cells. Exemplary blocking groups include but are not limited to methyl, O-methyl, and amine groups

According to a fourth embodiment, the present invention provides a polynucleotide that comprises a region that has a sequence substantially similar to: SEQ. ID No. 1, GUUGGAGCUGUUGGCGUAGUU (sense strand or sense region). More preferably the region is the same as SEQ. ID No. 1.

SEQ. ID No. 1 down regulates Kras genes that contain a G→T alteration at nucleotide 35 (codon 12, also referred to as a G12V allele) of the open reading frame, but not the wild type gene. Thus, it has no appreciable effect on the wild type gene. The bold U represents the SNP site. Mutations in the Kras gene have been associated with a wide variety of human cancers (see, for instance, Lee, S. H. (2003) “BRAF and KRAS mutations in stomach cancer” Oncogene 22(44):6942-5; Fong, K. M. et al., (1998) “KRAS codon 12 mutations in Australian non-small cell lung cancer” Aust. N. Z. J. Med. (2): 184-9). Thus, silencing of these genes is particularly desirable.

As the sequence of an siRNA can vary between 18-30 base pairs, it is important to note that in versions of the molecule that differ in length from those reported above, the identity of the nucleotides at the 5′ end of the antisense strand must be fixed to retain SNP-specific activity.

The sequence can be utilized in a variety of cell types including those of or derived from human lung, stomach, colon, endometrial, brain, breast, and others.

The sequence can be incorporated into a duplex siRNA having separate strands or unimolecular structure (modified or unmodified), and all conditions previously described as pertaining to modifications, size, methods of delivery, methods of detection, and applications are included within the spirit and scope of this embodiment.

The inventive polynucleotide of this embodiment and other embodiments should be understood as preferably being directed to polynucleotides that have been either chemically synthesized or enzymatically generated in vitro or in vivo through direct or indirect human manipulation, by for example, the introduction of a vector that codes for the polynucleotide.

According to a fifth embodiment, the present invention provides a method for down regulating the expression of a mutant form of the human Kras gene. This method comprises administering a siRNA of the fourth embodiment to a cell or organism that is expressing or is capable of expressing the target gene. Further, all conditions previously described pertaining to modifications, size, methods of delivery, methods of detection, and applications are included within the spirit and scope of this embodiment.

According to a sixth embodiment, the present invention is directed to a polynucleotide that comprises a region that has a sequence substantially similar to: SEQ. ID. No.2, 5′ GUUGGAGCUGGUGGCGUAGUU (sense strand or sense region), which exclusively down regulates wildtype Kras genes (G at position 35 of the open reading frame) but has no appreciable effect on the G→T (mutant) version of the gene. More preferably the region is the same as SEQ. ID No. 2. The sequence can be incorporated into a siRNA, and all conditions previously described pertaining to size, modifications, methods of delivery, methods of detection, and applications are included within the spirit and scope of this embodiment.

According to a seventh embodiment, the present invention provides a method for down regulating the expression of the wild type form of the human Kras gene. This method comprises administering a siRNA of the sixth embodiment to a cell or organism that is expressing or is capable of expressing the target gene. Further, all conditions previously listed pertaining to size, modifications, methods of delivery, methods of detection, and applications are included within the spirit and scope of this embodiment.

One preferred set of modifications that can be used for either SNP containing siRNA or non-SNP containing siRNA comprises an siRNA comprising: (a) a first 5′ terminal sense nucleotide and a second 5′ terminal sense nucleotide, wherein each of the first 5′ terminal sense nucleotide and the second 5′ terminal sense nucleotide comprises a 2′-O-alkyl modification; (b) a first 5′ terminal antisense nucleotide, wherein the first 5′ terminal antisense nucletide is phosphorylated at its 5′ position (i.e., having the structure R—CH₂—O—PO₄, wherein the —CH₂— is the 5′ CH₂ of a sugar moiety, preferably a ribosyl moiety, and R represents the remainder of the first 5′ terminal antisense nucleotide); and (c) a second 5′ terminal antisense nucleotide, wherein the second 5′ terminal antisense nucleotide comprises a 2′-O-alkyl modification. In a preferred embodiment, each of the 2′-O-alkyl modifications of the previous sentence are 2′-O-methyl modifications. This set of modifications is described in more detail in U.S. Provisional Patent Application Ser. No. 60/630,228, filed Nov. 22, 2004, and in U.S. patent application Ser. No. 11/019,831, filed Dec. 22, 2004, each of which is herein incorporated by reference. Specifically incorporated by reference are the following pages in U.S. patent application Ser. No. 11/019,831: pages 40-44 (describing synthesis of molecules with such modifications) and 23-38 (describing molecules and the benefits of particular modifications).

The polynucleotides of the present invention may be synthesized by any method that is now known or that comes to be known and that from reading this disclosure a person of ordinary skill in the art would appreciate would be useful to synthesize the molecules of the present invention.

For example, siRNA duplexes with two separate strands that contain the specified modifications may be chemically synthesized by synthesizing each of the strands using compositions of matter and methods described in Scaringe, S. A. (2000) “Advanced 5′-silyl-2′-orthoester approach to RNA oligonucleotide synthesis,” Methods Enzymol. 317, 3-18; Scaringe, S. A. (2001) “RNA oligonucleotide synthesis via 5′-silyl-2′-orthoester chemistry,” Methods 23, 206-217; Scaringe, S. and Caruthers, M. H. (1999), U.S. Pat. No. 5,889,136; Scaringe, S. and Caruthers, M. H. (1999), U.S. Pat. No. 6,008,400; Scaringe, S. (2000), U.S. Pat. No. 6,111,086; Scaringe, S. (2003) U.S. Pat. No. 6,590,093; each of which is incorporated herein by reference. (Similarly, unimoleclular polynucleotides can be synthesized by using these techniques but synthesizing only one longer strand.) The synthesis method utilizes nucleoside base-protected 5′-O-silyl-2′-O-orthoester-3′-O-phosphoramidites to assemble the desired unmodified siRNA sequence on a solid support in the 3′ to 5′ direction. Briefly, synthesis of the required phosphoramidites begins from standard base-protected ribonucleosides (uridine, N⁴-acetylcytidine, N²-isobutyrylguanosine and N⁶-isobutyryladenosine). Introduction of the 5′-O-silyl and 2′-O-orthoester protecting groups, as well as the reactive 3′-O-phosphoramidite moiety is then accomplished in five steps, including:
Simultaneous transient blocking of the 5′- and 3′-hydroxyl groups of the nucleoside sugar with Markiewicz reagent (1,3-dichloro-1,1,3,3,-tetraisopropyldisiloxane [TIPS-Cl₂]) in pyridine solution {Markiewicz, W. T. (1979) “Tetraisopropyldisiloxane-1,3-diyl, a Group for Simultaneous Protection of 3′- and 5′-Hydroxy Functions of Nucleosides,” J. Chem. Research(S), 24-25}, followed by chromatographic purification;
Regiospecific conversion of the 2′-hydroxyl of the TWPS-nucleoside sugar to the bis(acetoxyethyl)orthoester [ACE derivative] using tris(acetoxyethyl)-orthoformate in dichloromethane with pyridinium p-toluenesulfonate as catalyst, followed by chromatographic purification;
Liberation of the 5′- and 3′-hydroxyl groups of the nucleoside sugar by specific removal of the TIPS-protecting group using hydrogen fluoride and N,N,N″N′-tetramethylethylene diamine in acetonitrile, followed chromatographic purification;
Protection of the 5′-hydroxyl as a 5′-O-silyl ether using benzhydroxy-bis(trimethylsilyloxy)silyl chloride [BzH—Cl] in dichloromethane, followed by chromatographic purification; and
Conversion to the 3′-O-phosphoramidite derivative using bis(N,N-diisopropylamino)methoxyphosphine and 5-ethylthio-1H-tetrazole in dichloromethane/acetonitrile, followed by chromatographic purification.

The phosphoramidite derivatives are typically thick, colorless to pale yellow syrups. For compatibility with automated RNA synthesis instrumentation, each of the products is dissolved in a pre-determined volume of anhydrous acetonitrile, and this solution is aliquoted into the appropriate number of serum vials to yield a 1.0-mmole quantity of phosphoramidite in each vial. The vials are then placed in a suitable vacuum desiccator and the solvent removed under high vacuum overnight. The atmosphere is then replaced with dry argon, the vials are capped with rubber septa, and the packaged phosphoramidites are stored at −20° C. until needed. Each phosphoramidite is dissolved in sufficient anhydrous acetonitrile to give the desired concentration prior to installation on the synthesis instrument.

The synthesis of the desired oligoribonucleotide is carried out using automated synthesis instrumentation. It begins with the 3′-terminal nucleoside covalently bound via its 3′-hydroxyl to a solid beaded polystyrene support through a cleavable linkage. The appropriate quantity of support for the desired synthesis scale is measured into a reaction cartridge, which is then affixed to synthesis instrument. The bound nucleoside is protected with a 5′-O-dimethoxytrityl moiety, which is removed with anhydrous acid (3% [v/v] dichloroacetic acid in dichloromethane) in order to free the 5′-hydroxyl for chain assembly.

Subsequent nucleosides in the sequence to be assembled are sequentially added to the growing chain on the solid support using a four-step cycle, consisting of the following general reactions:
1. Coupling: the appropriate phosphoramidite is activated with 5-ethylthio-1H-tetrazole and allowed to react with the free 5′-hydroxyl of the support bound nucleoside or oligonucleotide. Optimization of the concentrations and molar excesses of these two reagents, as well as of the reaction time, results in coupling yields generally in excess of 98% per cycle.
2. Oxidation: the internucleotide linkage formed in the coupling step leaves the phosphorous atom in its P(III) [phosphite] oxidation state. The biologically-relevant oxidation state is P(V) [phosphate]. The phosphorous is therefore oxidized from P(III) to P(V) using a solution of tert-butylhydroperoxide in toluene.
3. Capping: the small quantity of residual un-reacted 5′-hydroxyl groups must be blocked from participation in subsequent coupling cycles in order to prevent the formation of deletion-containing sequences. This is accomplished by treating the support with a large excess of acetic anhydride and 1-methylimidazole in acetonitrile, which efficiently blocks residual 5′-hydroxyl groups as acetate esters.
4. De-silylation: the silyl-protected 5′-hydroxyl must be deprotected prior to the next coupling reaction. This is accomplished through treatment with triethylamine trihydrogen fluoride in N,N-dimethylformamide, which rapidly and specifically liberates the 5′-hydroxyl without concomitant removal of other protecting groups (2′-O-ACE, N-acyl base-protecting groups, or phosphate methyl).

It should be noted that in between the above four reaction steps are several washes with acetonitrile, which are employed to remove the excess of reagents and solvents prior to the next reaction step. The above cycle is repeated the necessary number of times until the unmodified portion of the oligoribonucleotide has been assembled. The above synthesis method is only exemplary and should not be construed as limited the means by which the molecules may be made. Any method that is now known or that comes to be known for synthesizing siRNA and that from reading this disclosure one skilled in the art would conclude would be useful in connection with the present invention may be employed.

The siRNA duplexes of certain embodiments of the present invention include two modified nucleosides (2′-O-methyl derivatives) at the 5′-end of each strand. The 5′-O-silyl-2′-O-methyl-3′-O-phosphoramidite derivatives required for the introduction of these modified nucleosides are prepared using procedures similar to those described previously (steps 4 and 5 above), starting from base-protected 2′-O-methyl nucleosides (2′-O-methyl-uridine, 2′-O-methyl-N⁴-acetylcytidine, 2′-O-methyl-N²-isobutyrylguanosine and 2′ -O-methyl-N⁶-isobutyryl adenosine). The absence of the 2′-hydroxyl in these modified nucleosides eliminates the need for ACE protection of these compounds. As such, introduction of the 5′-O-silyl and the reactive 3′-O-phosphoramidite moiety is accomplished in two steps, including:
1. Protection of the 5′-hydroxyl as a 5′-O-silyl ether using benzhydroxy-bis(trimethylsilyloxy)silyl chloride [BzH—Cl] in N,N-dimethylformamide, followed by chromatographic purification; and
2. Conversion to the 3′-O-phosphoramidite derivative using bis(N,N-diisopropylamino)methoxyphosphine and 5-ethylthio-1H-tetrazole in dichloromethane/acetonitrile, followed by chromatographic purification.

Post-purification packaging of the phosphoramidites is carried out using the procedures described previously for the standard nucleoside phosphoramidites. Similarly, the incorporation of the two 5′-O-silyl-2′-O-methyl nucleosides via their phosphoramidite derivatives is accomplished by twice applying the same four-step cycle described previously for the standard nucleoside phosphoramidites.

The siRNA duplexes of certain embodiments of this invention include a phosphate moiety at the 5′-end of the antisense strand. This phosphate is introduced chemically as the final coupling to the antisense sequence. The required phosphoramidite derivative (bis(cyanoethyl)-N,N-diisopropylamino phosphoramidite) is synthesized as follows in brief: phosphorous trichloride is treated one equivalent of N,N-diisopropylamine in anhydrous tetrahydrofuran in the presence of excess triethylamine. Then, two equivalents of 3-hydroxypropionitrile are added and allowed to react completely. Finally, the product is purified by chromatography. Post-purification packaging of the phosphoramidite is carried out using the procedures described previously for the standard nucleoside phosphoramidites. Similarly, the incorporation of the phosphoramidite at the 5′-end of the antisense strand is accomplished by applying the same four-step cycle described previously for the standard nucleoside phosphoramidites.

The modified, protected oligoribonucleotide remains linked to the solid support at the finish of chain assembly. A two-step rapid cleavage/deprotection procedure is used to remove the phosphate methyl protecting groups, cleave the oligoribonucleotide from the solid support, and remove the N-acyl base-protecting groups. It should be noted that this procedure also removes the cyanoethyl protecting groups from the 5′-phosphate on the antisense strand. Additionally, the procedure removes the acetyl functionalities from the ACE orthoester, converting the 2′-O-ACE protecting group into the bis(2-hydroxyethyl)orthoester. This new orthoester is significantly more labile to mild acid, as well as more hydrophilic than the parent ACE group. The two-step procedure is briefly as follows:
1. The support-bound oligoribonucleotide is treated with a solution of disodium 2-carbamoyl-2-cyanoethylene-1,1-dithiolate trihydrate in N,N-dimethylformamide. This reagent rapidly and efficiently removes the methyl protecting groups from the internucleotide phosphate linkages without cleaving the oligoribonucleotide from the solid support. The support is then washed with water to remove excess dithiolate.
2. The oligoribonucleotide is cleaved from the solid support with 40% (w/v) aqueous methylamine at room temperature. The methylamine solution containing the crude oligoribonucleotide is then heated to 55° C. to remove the protecting groups from the nucleoside bases. The crude orthoester-protected oligoribonucleotide is obtained following solvent removal in vacuo.

Removal of the 2′-orthoesters is the final step in the synthesis process. This is accomplished by treating the crude oligoribonucleotide with an aqueous solution of acetic acid and N,N,N′,N′-tetramethyl ethylene diamine, pH 3.8, at 55° C. for 35 minutes. The completely deprotected oligoribonucleotide is then desalted by ethanol precipitation and isolated by centrifugation.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified.

### EXAMPLES

### Example 1

### Mismatch Interference Analysis of a Luciferase siRNA to Identify Sensitive Positions within the Duplex

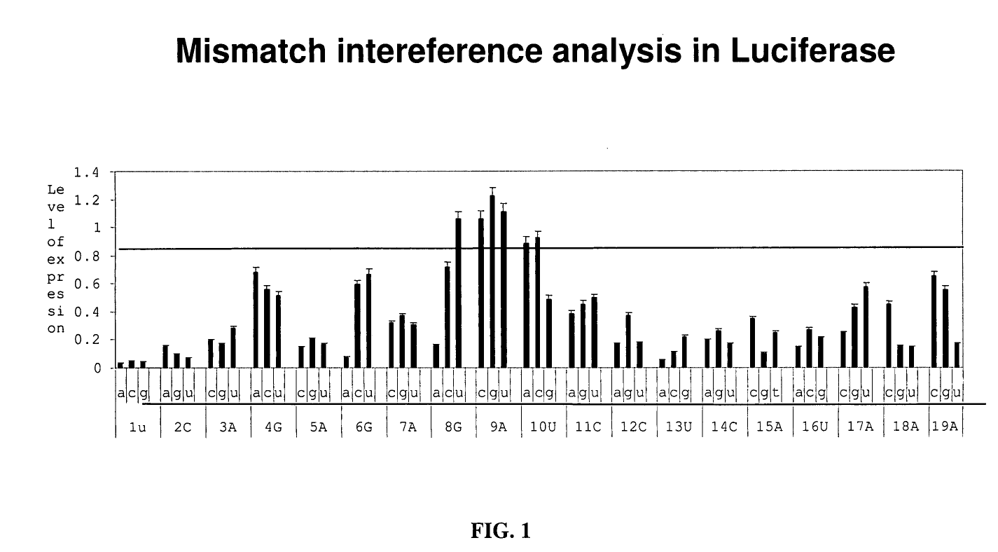

To identify key positions within siRNA duplexes that were sensitive to mismatches, 57 derivatives of the Luc-5 siRNA ( SEQ. ID NO. 46: 5′ TCAGAGAGATCCTCATAAA, sense strand) containing one of three base pair mismatches at each position of the duplex were synthesized using 2′-O-ACE chemistry. Subsequently, these molecules were transfected into HEK293 cells (along with a luciferase expression vector, pCMVluc) at 100 nM concentrations using Lipofectamine 2000 (Invitrogen, Carlsbad, Calif.). After 24-48 hours, the level of gene silencing was determined using the SteadyGlow assay (Promega, Madison, Wis.). Results in FIG. 1 show that introduction of base pair mismatches at position 9 consistently leads to a dramatic loss of silencing activity. Introduction of base pair mismatches at positions 8 and 10 also lead to loss of silencing activity, yet with a lesser frequency. (Sequences are provided with respect to the sense strand only; however, the unless otherwise indicated, the presence of the complementary antisense strand is implied.)

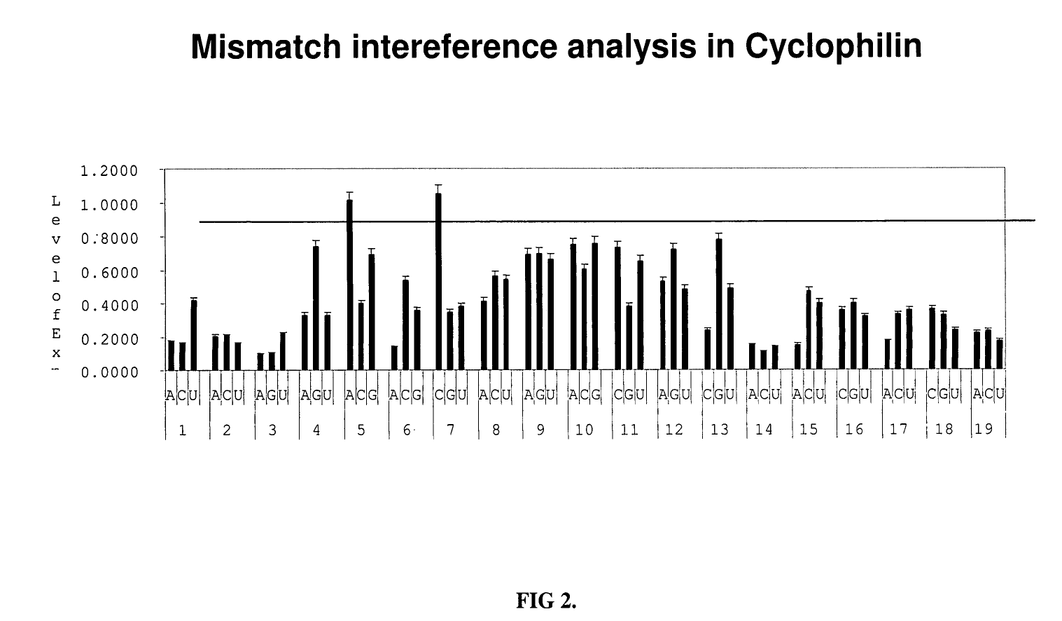

In FIGS. 1 and 2, the X-axis identifies the siRNA by the change in a particular nucleotide relative to the wild type siRNA at a particular position. The Y-axis shows the level of expression as compared to the wild type expression of the gene. A score of 1.0 means that there was no difference between expression in the presence of the particular siRNA that contains the variable site and in the absence of that siRNA (i.e., there was normal expression of the wild type gene). A score of e.g., 0.2 represents silencing of 80% of the target (expression at a level of 20%) as compared to normal expression of the target.

Similar procedures were performed on an siRNA directed against human cyclophilin B gene (FIG. 2) and showed that some of the positions of the duplex that were sensitive to mismatch repair extended from nucleotide 8-12.

### Example 2

### Performing a siRNA Walk to Identify a SNP-Specific siRNA

Identification of SNP-specific siRNA has been performed on a frequently observed variant of the h-Kras gene. To accomplish this, 19 unique siRNA, each overlapping the position of the SNP and shifted by a single basepair, were constructed against both the WT (NM_{—}004985, See Table I) and SNP-containing (GOT, Glycine 124Valine, G12V, see Table II) messages using Dharmacon's proprietary 2′-O-ACE chemistry. Duplexes were co-transfected into HEK293 cells (Lipofectamine 2000, Invitrogen) along with plasmid P1025a or P1025c (Biomyx Technology, San Diego, Calif.) that express either the WT or G12V-SNP-containing variety of the Kras, respectively. After 24 hours, the levels of WT or SNP-containing mRNA were quantitated by B-DNA (Genospectra).

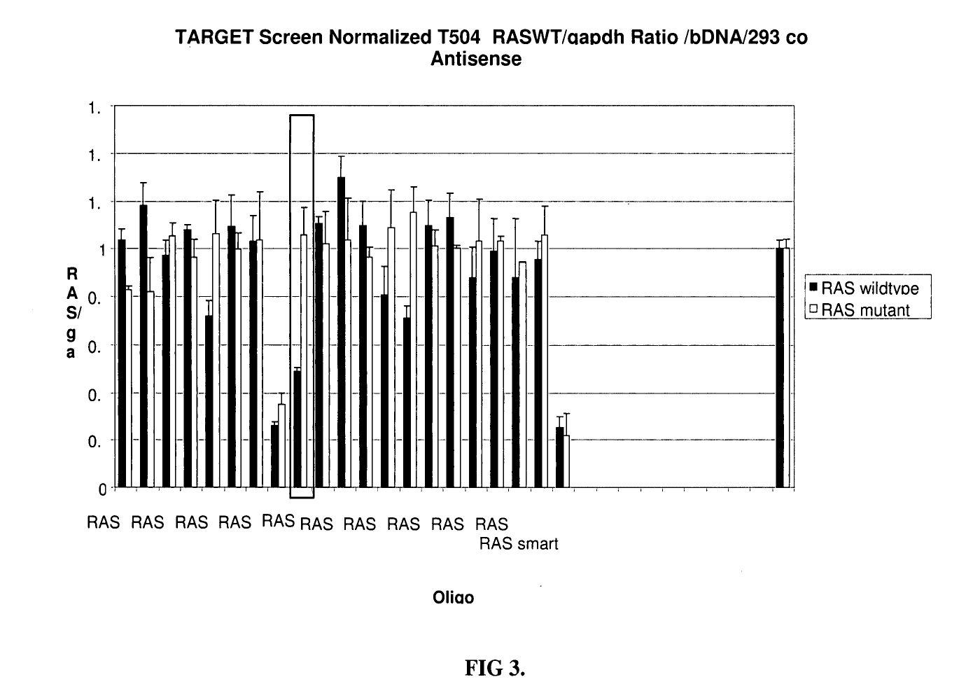

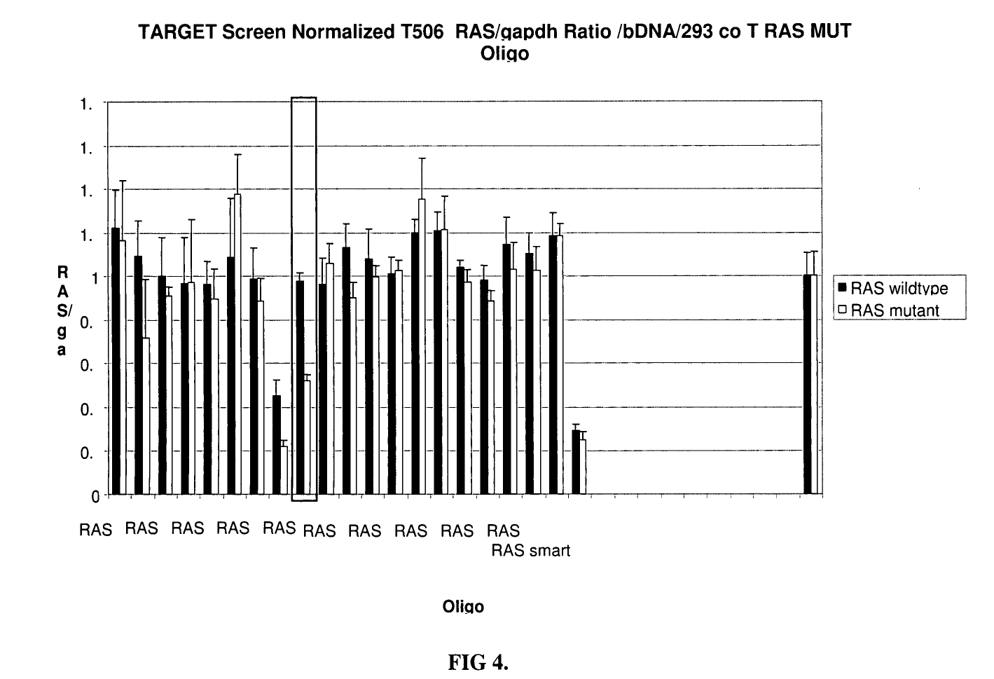

The results of these studies are presented in FIGS. 3 and 4 and identify: (a) a single siRNA (SEQ. ID NO. 2: 5′-GUUGGAGCUGGUGGCGUAGUU, sense strand) that knocks down the wildtype version of the gene but has little or no effect on the G12V mutant (see FIG. 3, boxed region); and (b) a single siRNA that knocks down the mutant version of the gene but has little or no effect on the wildtype transcript (FIG. 4, boxed region, SEQ. ID NO. 1, 5′ GUUGGAGCUGUUGGCGUAGUU, sense strand). The ability of both sequences to differentiate between mutant and wildtype forms supports the data generated in Example 1, which shows that base pair mismatches between an siRNA and a given target that are localized at position 9 eliminate silencing activity for that target.

Sequence of Wildtype and SNP-Containing Sequences:

Kras Wildtype (SEQ. ID No. 6)

NM_{—}004985

atgactgaat ataaacttgt ggtagttgga gctggtggcg
taggcaagag tgccttgacg atacagctaa ttcagaatca ttttgtggac
gaatatgatc caacaataga ggattcctac aggaagcaag tagtaattga
tggagaaacc tgtctcttgg atattctcga cacagcaggt caagaggagt
acagtgcaat gagggaccag tacatgagga ctggggaggg ctttctttgt
gtatttgcca taaataatac taaatcattt gaagatattc accattatag
agaacaaatt aaaagagtta aggactctga agatgtacct atggtcctag
taggaaataa atgtgatttg ccttctagaa cagtagacac aaaacaggct
caggacttag caagaagtta tggaattcct tttattgaaa catcagcaaa
gacaagacag ggtgttgatg atgccttcta tacattagtt cgagaaattc
gaaaacataa agaaaagatg agcaaagatg gtaaaaagaa gaaaaagaag
tcaaagacaa agtgtgtaat tatgtaa

Kras G12V Mutant (SEQ. ID No. 7)

atgactgaat ataaacttgt ggtagttgga gctgttggcg
taggcaagag tgccttgacg atacagctaa ttcagaatca ttttgtggac
gaatatgatc caacaataga ggattcctac aggaagcaag tagtaattga
tggagaaacc tgtctcttgg atattctcga cacagcaggt caagaggagt
acagtgcaat gagggaccag tacatgagga ctggggaggg ctttctttgt
gtatttgcca taaataatac taaatcattt gaagatattc accattatag
agaacaaatt aaaagagtta aggactctga agatgtacct atggtcctag
taggaaataa atgtgatttg ccttctagaa cagtagacac aaaacaggct
caggacttag caagaagtta tggaattcct tttattgaaa catcagcaaa
gacaagacag ggtgttgatg atgccttcta tacattagtt cgagaaattc
gaaaacataa agaaaagatg agcaaagatg gtaaaaaqaa gaaaaagaag
tcaaagacaa agtgtgtaat tatgtaa

TABLE I

| | | |
|---|---|---|
| | SEQ IN | SEQ. ID |
| Wild type oligo sequence | | NO. |

| | | |
|---|---|---|
| uugugguaguuggagcugguu | 1 | 8 |
| ugugguaguuggagcugguuu | 2 | 9 |
| gugguaguuggagcugguguu | 3 | 10 |
| ugguaguuggagcuggugguu | 4 | 11 |
| gguaguuggagcugguggcuu | 5 | 12 |
| guaguuggagcugguggcguu | 6 | 13 |
| uaguuggagcugguggcguuu | 7 | 14 |
| aguuggagcugguggcguauu | 8 | 15 |
| guuggagcugguggcguaguu | 9 | 2 |
| uuggagcugguggcguagguu | 10 | 16 |
| uggagcugguggcguaggcuu | 11 | 17 |
| ggagcugguggcguaggcauu | 12 | 18 |
| gagcugguggcguaggcaauu | 13 | 19 |
| agcugguggcguaggcaaguu | 14 | 20 |
| gcugguggcguaggcaagauu | 15 | 21 |
| cugguggcguaggcaagaguu | 16 | 22 |
| ugguggcguaggcaagaguuu | 17 | 23 |
| gguggcguaggcaagaguguu | 18 | 24 |
| guggcguaggcaagagugcuu | 19 | 25 |
| uggcguaggcaagagugccuu | 20 | 26 |

TABLE II

| | | |
|---|---|---|
| Mutant oligo sequence (Bold | SEQ. IN | SEQ. ID |
| “u” represents position of SNP) | | NO. |

| | | |
|---|---|---|
| uugugguaquuggagcuguuu | 1 | 27 |
| ugugguaguuggagcuguuuu | 2 | 28 |
| gugguaguuggagcuguuguu | 3 | 29 |
| ugguaguuggagcuguugguu | 4 | 30 |
| gguaguuggagcuguuggcuu | 5 | 31 |
| guaguuggagcuguuggcguu | 6 | 32 |
| uaguuggagcuguuggcguuu | 7 | 33 |
| aguuggagcuguuggcguauu | 8 | 34 |
| guuggagcuguuggcguaguu | 9 | 1 |
| uuggagcuguuggcguagguu | 10 | 35 |
| uggagcuguuggcguaggcuu | 11 | 36 |
| ggagcuguuggcguaggcauu | 12 | 37 |
| gagcuguuggcguaggcaauu | 13 | 38 |
| agcuguuggcguaggcaaguu | 14 | 39 |
| gcuguuggcguaggcaagauu | 15 | 40 |
| cuguuggcguaggcaagaguu | 16 | 41 |
| uguuggcguaggcaagaguuu | 17 | 42 |
| guuggcguaggcaagaguguu | 18 | 43 |
| uuggcguaggcaagagugcuu | 19 | 44 |
| uggcguaggcaagagugccuu | 20 | 45 |

Table I and II report the sequence of the sense strand of each siRNA synthesized. The “UU” dinucleotide overhang is added to the 3′ end of each sequence.

### Example 3

Improvement of siRNA Functionality Using 2′-O-Methyl Modifications of Positions 1 and 2 of the Sense Strand

To improve the functionality of SEQ. ID NO. 1 and SEQ. ID NO. 2, the 2′ carbon of the ribose ring of nucleotides 1 and 2 or 1, 2, and 3 of the sense strand of siRNA (using either two separate strands or shRNA) could be made incorporating these sequences and modified to contain 2′-O-methyl groups. Subsequently, duplexes (or equivalent unimolecular structures) could be transfected into HEK293 cells expressing the appropriate reporter construct and cultured for 48 hours. To test the ability of these modifications to improve the level of silencing, total RNA could be prepared from each culture (and relevant controls) and assayed using the branched DNA assay.

1. A method of identifying SNP specific siRNA or non-SNP containing siRNA, said method comprising:
(a) comparing the silencing effect of:
(i) at least two SNP containing siRNA in cells that contain a SNP target sequence,
(ii) said at least two SNP containing siRNA in cells that contain a wild type target sequence,
(iii) at least two non-SNP containing siRNA in cells that contain a SNP target sequence, and
(iv) said at least two non-SNP containing siRNA in cells that contain a wild type target sequence; and
(b) identifying a SNP specific siRNA that silences said SNP containing target sequence, but does not silence said wild type target sequence, or identifying a non-SNP containing siRNA that silences said wild type target sequence, but does not silence said SNP target sequence.

2. The method of claim 1, wherein said comparing of either or both of said SNP containing target sequence and said wild type sequence is measured through monitoring expression of a reporter expression construct or a target gene expressed from an expression vector.

3. A method of gene silencing comprising introducing a SNP specific siRNA that silences said SNP containing target sequence, but does not silence a wild type target sequence, wherein said SNP specific siRNA comprises a sense strand and an antisense strand that are capable of forming a duplex of 18-30 base pairs.

4. The method of claim 3, wherein said SNP specific siRNA comprises:
(a) a first 5′ terminal sense nucleotide and a second 5′ terminal sense nucleotide, wherein each of said first 5′ terminal sense nucleotide and said second 5′ terminal sense nucleotide comprises a 2′-O-alkyl group; and
(b) a first 5′ terminal antisense nucleotide, wherein said first 5′ terminal antisense nucleotide is phosphorylated at said first 5′ terminal antisense nucleotide's 5′ carbon position.

5. The method of claim 4, wherein said SNP specific siRNA further comprises a third 5′ terminal sense nucleotide and said third 5′ terminal sense nucleotide comprises a 2′-O-alkyl group.

6. The method of claim 4, wherein said SNP specific siRNA further comprises a second 5′ terminal antisense nucleotide, wherein each of said first 5′ terminal antisense nucleotide and said second 5′ terminal antisense nucleotide comprises a 2′-O-alkyl group.

7. The method of claim 6, wherein said SNP specific siRNA further comprises a third 5′ terminal antisense nucleotide and said third 5′ terminal sense nucleotide comprises a 2′-O-alkyl group.

8. The method of claim 4, further comprising a second 5′ terminal antisense nucleotide, wherein said second 5′ terminal antisense nucleotide comprises a 2′-O-methyl modification, and wherein said 2′-O-alkyl group is a 2′-O-methyl group.

9. The method of claim 4, wherein said SNP specific siRNA further comprises at least one additional 2′-O-alkyl modification on or more Cs or Us of the sense strand.

10. The method of claim 4, wherein said SNP specific siRNA further comprises at least one fluorine modification on or more Cs or Us of the antisense strand.

11. The method of claim 6, wherein said SNP specific siRNA further comprises at least one additional 2′-O-alkyl modification on or more Cs or Us of the sense strand.

12. The method of claim 6, wherein said SNP specific siRNA further comprises at least one Fl modification on or more Cs or Us of the antisense strand.

13. The method of claim 4, wherein said SNP containing target sequence comprises at least one base pair mismatch near the 5′ end of the antisense strand.

14. The method of claim 4, wherein said each 2′-O-alkyl modification is a 2′-O-methyl modification.

15. The method of claim 6, wherein said each 2′-O-alkyl modification is a 2′-O-methyl modification.

16. A method of gene silencing comprising introducing a wild type siRNA that silences a wild type containing target sequence, but does not silence a SNP specific containing target sequence, wherein said wild type siRNA comprises a sense strand and an antisense strand that are capable of forming a duplex of 18-30 base pairs.

17. The method of claim 15, wherein said wild type siRNA comprises:
(a) a first 5′ terminal sense nucleotide and a second 5′ terminal sense nucleotide, wherein each of said first 5′ terminal sense nucleotide and said second 5′ terminal sense nucleotide comprises a 2′-O-alkyl group; and
(b) a first 5′ terminal antisense nucleotide, wherein said first 5′ terminal antisense nucleotide is phosphorylated at said first 5′ terminal antisense nucleotide's 5′ carbon position.

18. The method of claim 16, wherein said wild type siRNA further comprises a third 5′ terminal sense nucleotide and said third 5′ terminal sense nucleotide comprises a 2′-O-alkyl group.

19. The method claim 16, wherein said wild type siRNA further comprises a second 5′ terminal antisense nucleotide, wherein each of said first 5′ terminal antisense nucleotide comprising and second 5′ terminal antisense nucleotide comprises a 2′-O-alkyl group.

20. The method of claim 16, wherein said wild type siRNA further comprises a third 5′ terminal antisense nucleotide and said third 5′ terminal antisense nucleotide comprises a 2′-O-alkyl group.

21. The method of claim 18, wherein said wild type siRNA further comprises a third 5′ terminal antisense nucleotide and said third 5′ terminal antisense nucleotide comprises a 2′-O-alkyl group.

22. The method of claim 16, wherein said wild type siRNA further comprises at least one additional 2′-O-alkyl modification on or more Cs or Us of the sense strand.

23. The method of claim 16, wherein said wild type siRNA further comprises at least one Fl modification on or more Cs or Us of the antisense strand.

24. The method of claim 18, wherein said wild type siRNA further comprises at least one additional 2′-O-alkyl modification on or more Cs or Us of the sense strand.

25. The method of claim 18, wherein said wild type siRNA further comprises at least one Fl modification on or more Cs or Us of the antisense strand.

26. The method of claim 16, wherein said SNP containing target sequence comprises at least one base pair mismatch near the 5′ end of the antisense strand.

27. The method of claim 16, wherein said each 2′-O-alkyl modification is a 2′-O-methyl modification.

28. A polynucleotide, wherein the polynucleotide comprises a region that has a sequence substantially similar to: SEQ. ID. No. 1, GUUGGAGCUGUUGGCGUAGUU and said region forms part of a duplex that is 18-30 base pairs in length.

29. The polynucleotide of claim 27, wherein said sequence is the same as SEQ. ID. No. 1.

30. A method of silencing a SNP variant of the Kras gene, said method comprising introducing the polynucleotide of claim 27 into a cell.

31. A polynucleotide, wherein the polynucleotide comprises a region that has a sequence substantially similar to SEQ. ID No. 2, GUUGGAGCUGGUGGCGUAGUU and said region forms part of a duplex that is 18-30 base pairs in length.

32. The polynucleotide of claim 30, wherein said sequence is the same as SEQ. ID. No. 2.

33. A method of silencing the wild type Kras gene, said method comprising introducing the polynucleotide of claim 30 into a cell.


SEQUENCE LISTING (46 sequences)

SEQ ID NO: 1 - RNA, 21 nt, Homo sapiens
guuggagcuguuggcguaguu

SEQ ID NO: 2 - RNA, 21 nt, Homo sapiens
guuggagcugguggcguaguu

SEQ ID NO: 3 - RNA, 4 nt, Homo sapiens
uucg

SEQ ID NO: 4 - RNA, 9 nt, Homo sapiens
uuuguguag

SEQ ID NO: 5 - RNA, 10 nt, Homo sapiens
cuuccuguca

SEQ ID NO: 6 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 7 - DNA, 567 bp, Homo sapiens
atgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 8 - RNA, 21 nt, Homo sapiens
uugugguaguuggagcugguu

SEQ ID NO: 9 - RNA, 21 nt, Homo sapiens
ugugguaguuggagcugguuu

SEQ ID NO: 10 - RNA, 21 nt, Homo sapiens
gugguaguuggagcugguguu

SEQ ID NO: 11 - RNA, 21 nt, Homo sapiens
ugguaguuggagcuggugguu

SEQ ID NO: 12 - RNA, 21 nt, Homo sapiens
gguaguuggagcugguggcuu

SEQ ID NO: 13 - RNA, 21 nt, Homo sapiens
guaguuggagcugguggcguu

SEQ ID NO: 14 - RNA, 21 nt, Homo sapiens
uaguuggagcugguggcguuu

SEQ ID NO: 15 - RNA, 21 nt, Homo sapiens
aguuggagcugguggcguauu

SEQ ID NO: 16 - RNA, 21 nt, Homo sapiens
uuggagcugguggcguagguu

SEQ ID NO: 17 - RNA, 21 nt, Homo sapiens
uggagcugguggcguaggcuu

SEQ ID NO: 18 - RNA, 21 nt, Homo sapiens
ggagcugguggcguaggcauu

SEQ ID NO: 19 - RNA, 21 nt, Homo sapiens
gagcugguggcguaggcaauu

SEQ ID NO: 20 - RNA, 21 nt, Homo sapiens
agcugguggcguaggcaaguu

SEQ ID NO: 21 - RNA, 21 nt, Homo sapiens
gcugguggcguaggcaagauu

SEQ ID NO: 22 - RNA, 21 nt, Homo sapiens
cugguggcguaggcaagaguu

SEQ ID NO: 23 - RNA, 21 nt, Homo sapiens
ugguggcguaggcaagaguuu

SEQ ID NO: 24 - RNA, 21 nt, Homo sapiens
gguggcguaggcaagaguguu

SEQ ID NO: 25 - RNA, 21 nt, Homo sapiens
guggcguaggcaagagugcuu

SEQ ID NO: 26 - RNA, 21 nt, Homo sapiens
uggcguaggcaagagugccuu

SEQ ID NO: 27 - RNA, 21 nt, Homo sapiens
uugugguaguuggagcuguuu

SEQ ID NO: 28 - RNA, 21 nt, Homo sapiens
ugugguaguuggagcuguuuu

SEQ ID NO: 29 - RNA, 21 nt, Homo sapiens
gugguaguuggagcuguuguu

SEQ ID NO: 30 - RNA, 21 nt, Homo sapiens
ugguaguuggagcuguugguu

SEQ ID NO: 31 - RNA, 21 nt, Homo sapiens
gguaguuggagcuguuggcuu

SEQ ID NO: 32 - RNA, 21 nt, Homo sapiens
guaguuggagcuguuggcguu

SEQ ID NO: 33 - RNA, 21 nt, Homo sapiens
uaguuggagcuguuggcguuu

SEQ ID NO: 34 - RNA, 21 nt, Homo sapiens
aguuggagcuguuggcguauu

SEQ ID NO: 35 - RNA, 21 nt, Homo sapiens
uuggagcuguuggcguagguu

SEQ ID NO: 36 - RNA, 21 nt, Homo sapiens
uggagcuguuggcguaggcuu

SEQ ID NO: 37 - RNA, 21 nt, Homo sapiens
ggagcuguuggcguaggcauu

SEQ ID NO: 38 - RNA, 21 nt, Homo sapiens
gagcuguuggcguaggcaauu

SEQ ID NO: 39 - RNA, 21 nt, Homo sapiens
agcuguuggcguaggcaaguu

SEQ ID NO: 40 - RNA, 21 nt, Homo sapiens
gcuguuggcguaggcaagauu

SEQ ID NO: 41 - RNA, 21 nt, Homo sapiens
cuguuggcguaggcaagaguu

SEQ ID NO: 42 - RNA, 21 nt, Homo sapiens
uguuggcguaggcaagaguuu

SEQ ID NO: 43 - RNA, 21 nt, Homo sapiens
guuggcguaggcaagaguguu

SEQ ID NO: 44 - RNA, 21 nt, Homo sapiens
uuggcguaggcaagagugcuu

SEQ ID NO: 45 - RNA, 21 nt, Homo sapiens
uggcguaggcaagagugccuu

SEQ ID NO: 46 - DNA, 19 bp, Homo sapiens
tcagagagatcctcataaa